(19) United States
(12) Patent Application Publication
(10) Pub. No.: US 2026/0056067 A1
(43) Pub. Date: Feb. 26, 2026

(54) WATER TEMPERATURE SENSOR

(71) Applicant: Torc Robotics, Inc., Blacksburg, VA (US)

(72) Inventors: Garrett Madsen, Austin, TX (US); Joseph R. Fox-Rabinovitz, Austin, TX (US)

(21) Appl. No.: 18/810,179

(22) Filed: Aug. 20, 2024

(51) Int. Cl. G01K 13/02; B60S 1/48; G01N 25/62; G01N 33/18

(52) U.S. Cl. CPC G01K 13/026; B60S 1/485; G01N 25/62; G01N 33/1873

(57) ABSTRACT

The present application discloses systems and methods for detecting a frost condition using a water temperatures sensor on an autonomous vehicle. The water temperature sensor includes a wind tunnel tube forming a hollow path for an airflow. The system further includes a fluid valve coupled to a fluid reservoir to dispense a fluid into the airflow of the hollow path. The water temperature further includes wet-bulb temperature sensor affixed to a vent coupled to a distal end of the wind tunnel tube. The wet-bulb temperature sensor configured to capture sensor data indicating a frost condition.

### TECHNICAL FIELD

The field of the disclosure relates generally to sensors on an autonomous vehicle and, more specifically, a water temperature sensor on an autonomous vehicle for detecting frost conditions.

### BACKGROUND OF THE INVENTION

Autonomous vehicles are configured to operate in all weather conditions. These vehicles rely on sophisticated systems and sensors to detect objects and conditions to enable them to navigate roads with a high degree of autonomy. These vehicles typically identify objects and respond to them based on information from sensors on the vehicles. However, the ability for the sensors to provide accurate data can be greatly impacted by weather conditions, such as frost. Further, detecting these weather conditions is challenging for the conventional sensors on the autonomous vehicle. This limitation underscores the need for a water temperature sensor to detect when a frost condition occurs.

This section is intended to introduce the reader to various aspects of art that may be related to various aspects of the present disclosure described or claimed below. This description is believed to be helpful in providing the reader with background information to facilitate a better understanding of the various aspects of the present disclosure. Accordingly, it should be understood that these statements are to be read in this light and not as admissions of prior art.

### SUMMARY OF THE INVENTION

In one aspect, a system for a water temperature sensor for an autonomous vehicle is provided. The water temperature sensor includes a wind tunnel tube forming a hollow path for an airflow. The water temperature sensor further includes a fluid valve coupled to a fluid reservoir, the fluid valve is fluidly coupled with a proximal end of the wind tunnel tube to dispense a fluid into the airflow of the hollow path. The water temperatures sensor further includes a vent coupled to a distal end of the wind tunnel tube and a wet-bulb temperature sensor affixed to the vent.

In another aspect, an autonomous vehicle including a water temperature is provided. The water temperature sensor includes a wind tunnel tube forming a hollow path for an airflow, a fluid valve coupled to a fluid reservoir, the fluid valve fluidly coupled with a proximal end of the wind tunnel tube to dispense a fluid into the airflow of the hollow path. The water temperature sensor further includes a vent coupled to a distal end of the wind tunnel tube, and a wet-bulb temperature sensor affixed to the vent. The autonomous vehicle includes a dry-bulb temperature sensor. The autonomous vehicle also includes an autonomy computing system. The autonomy computing system includes a processor connected to a memory storing computer executable instructions, the processor, upon executing the computer executable instructions, is configured to: receive sensor data from the dry-bulb temperature sensor on the autonomous vehicle; detect the sensor data is below a temperature threshold; control the fluid valve on the water temperature sensor to dispense a fluid into the airflow of the hollow path; receive additional sensor data from the water temperature sensor, detect a frost condition from the additional sensor data; and initiate transmission of an indication of the frost condition to the autonomy computing system.

In yet another aspect, the disclosed computer-implemented method for detecting a frost condition on an autonomous vehicle is provided. The computer-implemented method also includes receiving sensor data from a dry-bulb temperature sensor, the sensor data representing a temperature value. The computer-implemented method further includes detecting the temperature value is below a temperature threshold; controlling a fluid valve coupled to a fluid reservoir, the fluid valve fluidly coupled with a proximal end of a wind tunnel tube of a water temperature sensor to dispense a fluid into the wind tunnel tube upon detection of the temperature value below the temperature threshold; receiving additional sensor data from a wet-blub temperature sensor; detecting a frost condition from the additional sensor data from the wet-bulb temperature sensor; and initiating transmission of an indication of the frost condition to the autonomy computing system.

Various refinements exist of the features noted in relation to the above-mentioned aspects. Further features may also be incorporated in the above-mentioned aspects as well.

These refinements and additional features may exist individually or in any combination. For instance, various features discussed below in relation to any of the illustrated examples may be incorporated into any of the above-described aspects, alone or in any combination.

### BRIEF DESCRIPTION OF DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 is a schematic diagram of an autonomous vehicle;

FIG. 2 is a block diagram of an autonomous vehicle;

FIG. 3 is an illustration of one embodiment of a water temperature sensor;

FIG. 4 is an illustration of one embodiment of a water temperature sensor on an autonomous vehicle;

FIG. 5 is a flow diagram of an example method of detecting a frost condition with a water temperature sensor; and

FIG. 6 is a block diagram of an example computing device.

Corresponding reference characters indicate corresponding parts throughout the several views of the drawings. Although specific features of various examples may be shown in some drawings and not in others, this is for convenience only. Any feature of any drawing may be referenced or claimed in combination with any feature of any other drawing. The drawings are not to scale unless otherwise noted.

### DETAILED DESCRIPTION

The following detailed description and examples set forth preferred materials, components, and procedures used in accordance with the present disclosure. This description and these examples, however, are provided by way of illustration only, and nothing therein shall be deemed to be a limitation upon the overall scope of the present disclosure.

The disclosed systems and methods are described, for clarity, using certain terminology when referring to and describing relevant components within the disclosure. Where possible, common industry terminology is employed in a manner consistent with its accepted meaning. Unless otherwise stated, such terminology should be given a broad interpretation consistent with the context of the present application and the scope of the appended claims.

The present disclosure is directed to a water temperature sensor on an autonomous vehicle. The water temperature sensor is configured to detect a frost condition impacting the autonomous vehicle. When a frost condition is detected, the water temperature sensor transmits an indication to an autonomy computing system of the autonomous vehicle. In some embodiments, the autonomy computing system is configured to compute a sensor degradation parameter for another sensor on the autonomous vehicle affected by the frost condition. The degradation parameter is based on the indication of the frost condition received from the water temperature sensor.

The water temperature sensor includes a wind tunnel tube forming a hollow path for the airflow. In some embodiments, a proximal end of the wind tunnel tube extends to the exterior of the vehicle. Embodiments of the water temperature sensor also include a fan located at the proximal end of the wind tunnel tube. The fan is configured to modify airflow through the hollow tube in response to external weather conditions observed by the autonomous vehicle.

The water temperature sensor includes a fluid valve. The fluid valve is coupled to the proximal end of the wind tunnel tube and a fluid reservoir. In some embodiments, the fan is disposed proximal to the fluid valve on the hollow path for the airflow. The fluid valve is configured to dispense a fluid from the fluid reservoir into the airflow of the hollow path of the wind tunnel tube. For example, the fluid reservoir includes a windshield washer reservoir. In some embodiments, the fluid has a freezing point less than 32° Fahrenheit. The fluid valve is configured to dispense the fluid into the hollow tube based on a humidity level of an external environment in which the autonomous vehicle is operating.

The water temperature sensor includes a vent coupled to a distal end of the wind tunnel tube. In various embodiments, a wet-bulb temperature sensor is affixed to the vent. The vent disrupts the path of the airflow to contact the fluid dispensed into the hollow path. The wet-bulb temperature sensor is configured to capture sensor data indicating temperature resulting from evaporative cooling of the fluid dispensed into the path of the airflow that contacts the vent.

In various embodiments, the water temperature sensor includes a processor connected to a memory storing computer executable instructions. The processor is configured to execute computer executable instructions from the memory. For example, the processor is configured to receive sensor data from the wet-bulb temperature sensor. The sensor data includes the temperature measured by the wet-bulb temperature sensor caused by the evaporative cooling of the fluid travelling along the airflow path that contacts the vent. The processor is further configured to identify a frost condition from the sensor data. The frost condition corresponds to weather conditions where sensor performance on the autonomous vehicle degrades resulting from the frost condition. In various embodiments, the processor is configured to transmit an indication of the frost condition to the autonomy computing system.

In various embodiments, the water temperature sensor is coupled to the autonomous vehicle. The water temperature sensor is further coupled to the autonomy computing system of the autonomous vehicle. The dry-bulb temperatures sensor is configured to capture sensor data indicating a temperature value. The temperature value includes the ambient temperature of the environment surrounding the autonomous vehicle. The autonomy computing system is configured to receive sensor data from the dry-bulb temperature sensor on the autonomous vehicle. The autonomy computing system processes the sensor data from the dry-bulb temperature sensor to detect a temperature below a temperature threshold. The temperature threshold corresponds to an ambient temperature where there is a possibility for frost to begin to form on sensors of the autonomous vehicle.

In various embodiments, the autonomy computing system is configured to control the fluid valve on the water temperature sensor when the sensor data indicates a temperature below the temperature threshold. The autonomy computing system is configured to control the fluid valve to dispense the fluid into the airflow of the hollow path. In some embodiments, the autonomy computing system controls the fluid valve to dispense the fluid into the airflow at a rate to match an external humidity condition of the autonomous vehicle.

The autonomy computing system is further configured to receive additional sensor data from the water temperature sensor. The additional temperature sensor data includes the sensor data captured by the water temperature sensor, such as the temperature data from the wet-bulb temperature sensor. The autonomy computing system is configured to process the additional sensor data to detect a frost condition from the additional sensor data. Upon detection of the frost condition, the autonomy computing system initiates transmission of an indication of the frost condition throughout the autonomy computing system. For example, the indication is transmitted to the calibration, mapping, motion estimation, perception and understanding, behaviors and planning, and control modules of the autonomy computing system.

In some embodiments, the autonomy computing system is configured to disengage the fluid valve upon the detection of the frost condition. The autonomy computing system is further configured to control the fluid valve to dispense the fluid on a periodic time interval to verify the frost condition from further sensor data from the water temperature sensor.

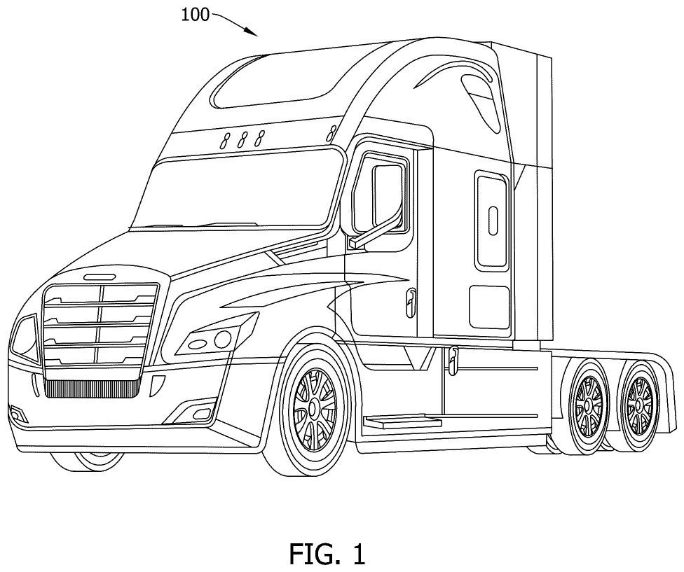

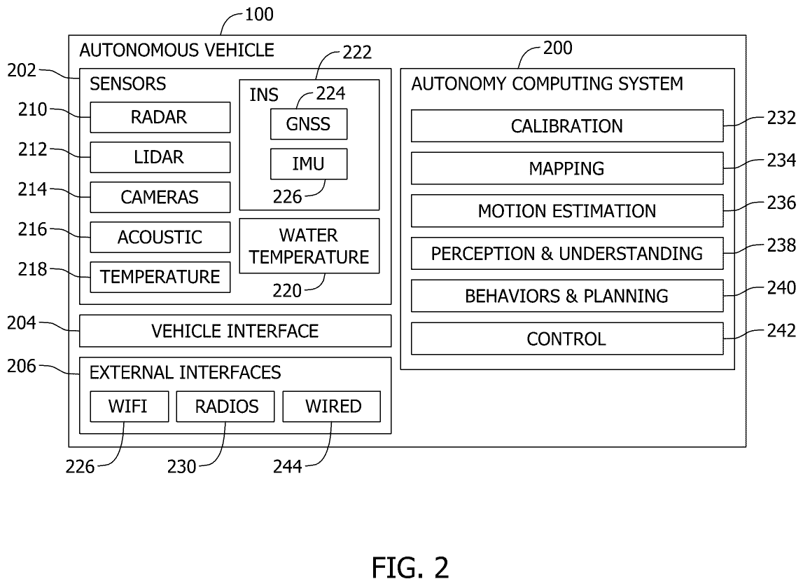

FIG. 1 is a schematic diagram of an autonomous vehicle 100. FIG. 2 is a block diagram of autonomous vehicle 100 shown in FIG. 1. In the example embodiment, autonomous vehicle 100 includes autonomy computing system 200, sensors 202, a vehicle interface 204, and external interfaces 206.

In the example embodiment, sensors 202 may include various sensors such as, for example, radio detection and ranging (RADAR) sensors 210, light detection and ranging (LiDAR) sensors 212, cameras 214, acoustic sensors 216, temperature sensors 218, water temperature sensor 220, or inertial navigation system (INS) 222, which may include one or more global navigation satellite system (GNSS) receivers 224 and one or more inertial measurement units (IMU) 226. Other sensors 202 not shown in FIG. 2 may include, for example, acoustic (e.g., ultrasound), internal vehicle sensors, meteorological sensors, or other types of sensors. Sensors 202 generate respective output signals based on detected physical conditions of autonomous vehicle 100 and its proximity. As described in further detail below, these signals may be used by autonomy computing system 120 to determine how to control operation of autonomous vehicle 100.

Cameras 214 are configured to capture images of the environment surrounding autonomous vehicle 100 in any aspect or field of view (FOV). The FOV can have any angle or aspect such that images of the areas ahead of, to the side, behind, above, or below autonomous vehicle 100 may be captured. In some embodiments, the FOV may be limited to particular areas around autonomous vehicle 100 (e.g., forward of autonomous vehicle 100, to the sides of autonomous vehicle 100, etc.) or may surround 360 degrees of autonomous vehicle 100. In some embodiments, autonomous vehicle 100 includes multiple cameras 214, and the images from each of the multiple cameras 214 may be stitched or combined to generate a visual representation of the multiple cameras'FOVs, which may be used to, for example, generate a bird's eye view of the environment surrounding autonomous vehicle 100. In some embodiments, the image data generated by cameras 214 may be sent to autonomy computing system 200 or other aspects of autonomous vehicle 100, and this image data may include autonomous vehicle 100 or a generated representation of autonomous vehicle 100. In some embodiments, one or more systems or components of autonomy computing system 200 may overlay labels to the features depicted in the image data, such as on a raster layer or other semantic layer of a high-definition (HD) map.

LiDAR sensors 212 generally include a laser generator and a detector that send and receive a LiDAR signal such that LiDAR point clouds (or “LiDAR images”) of the areas ahead of, to the side, behind, above, or below autonomous vehicle 100 can be captured and represented in the LiDAR point clouds. Radar sensors 210 may include short-range RADAR (SRR), mid-range RADAR (MRR), long-range RADAR (LRR), or ground-penetrating RADAR (GPR). One or more sensors may emit radio waves, and a processor may process received reflected data (e.g., raw radar sensor data) from the emitted radio waves. In some embodiments, the system inputs from cameras 214, radar sensors 210, or LiDAR sensors 212 may be fused or used in combination to determine conditions (e.g., locations of other objects) around autonomous vehicle 100.

GNSS receiver 224 is positioned on autonomous vehicle 100 and may be configured to determine a location of autonomous vehicle 100, which it may embody as GNSS data, as described herein. GNSS receiver 224 may be configured to receive one or more signals from a global navigation satellite system (e.g., Global Positioning System (GPS) constellation) to localize autonomous vehicle 100 via geolocation. In some embodiments, GNSS receiver 224 may provide an input to or be configured to interact with, update, or otherwise utilize one or more digital maps, such as an HD map (e.g., in a raster layer or other semantic map). In some embodiments, GNSS receiver 224 may provide direct velocity measurement via inspection of the Doppler effect on the signal carrier wave. Multiple GNSS receivers 224 may also provide direct measurements of the orientation of autonomous vehicle 100. For example, with two GNSS receivers 224, two attitude angles (e.g., roll and yaw) may be measured or determined. In some embodiments, autonomous vehicle 100 is configured to receive updates from an external network (e.g., a cellular network). The updates may include one or more of position data (e.g., serving as an alternative or supplement to GNSS data), speed/direction data, orientation or attitude data, traffic data, weather data, or other types of data about autonomous vehicle 100 and its environment.

IMU 226 is a micro-electrical-mechanical (MEMS) device that measures and reports one or more features regarding the motion of autonomous vehicle 100, although other implementations are contemplated, such as mechanical, fiber-optic gyro (FOG), or FOG-on-chip (SiFOG) devices. IMU 226 may measure an acceleration, angular rate, and or an orientation of autonomous vehicle 100 or one or more of its individual components using a combination of accelerometers, gyroscopes, or magnetometers. IMU 226 may detect linear acceleration using one or more accelerometers and rotational rate using one or more gyroscopes and attitude information from one or more magnetometers. In some embodiments, IMU 226 may be communicatively coupled to one or more other systems, for example, GNSS receiver 224 and may provide input to and receive output from GNSS receiver 224 such that autonomy computing system 200 is able to determine the motive characteristics (acceleration, speed/direction, orientation/attitude, etc.) of autonomous vehicle 100.

In the example embodiment, autonomy computing system 200 employs vehicle interface 204 to send commands to the various aspects of autonomous vehicle 100 that actually control the motion of autonomous vehicle 100 (e.g., engine, throttle, steering wheel, brakes, etc.) and to receive input data from one or more sensors 202 (e.g., internal sensors). External interfaces 206 are configured to enable autonomous vehicle 100 to communicate with an external network via, for example, a wired or wireless connection, such as Wi-Fi 228 or other radios 230. In embodiments including a wireless connection, the connection may be a wireless communication signal (e.g., Wi-Fi, cellular, LTE, 5g, Bluetooth, etc.).

In some embodiments, external interfaces 206 may be configured to communicate with an external network via a wired connection 244, such as, for example, during testing of autonomous vehicle 100 or when downloading mission data after completion of a trip. The connection(s) may be used to download and install various lines of code in the form of digital files (e.g., HD maps), executable programs (e.g., navigation programs), and other computer-readable code that may be used by autonomous vehicle 100 to navigate or otherwise operate, either autonomously or semi-autonomously. The digital files, executable programs, and other computer readable code may be stored locally or remotely and may be routinely updated (e.g., automatically or manually) via external interfaces 206 or updated on demand. In some embodiments, autonomous vehicle 100 may deploy with all of the data it needs to complete a mission (e.g., perception, localization, and mission planning) and may not utilize a wireless connection or other connection while underway.

In the example embodiment, autonomy computing system 200 is implemented by one or more processors and memory devices of autonomous vehicle 100. Autonomy computing system 200 includes modules, which may be hardware components (e.g., processors or other circuits) or software components (e.g., computer applications or processes executable by autonomy computing system 200), configured to generate outputs, such as control signals, based on inputs received from, for example, sensors 202. These modules may include, for example, a calibration module 232, a mapping module 234, a motion estimation module 236, a perception and understanding module 238, a behaviors and planning module 240, and a control module or controller 242, These modules may be implemented in dedicated hardware such as, for example, an application specific integrated circuit (ASIC), field programmable gate array (FPGA), or microprocessor, or implemented as executable software modules, or firmware, written to memory and executed on one or more processors onboard autonomous vehicle 100.

Autonomy computing system 200 of autonomous vehicle 100 may be completely autonomous (fully autonomous) or semi-autonomous. In one example, autonomy computing system 200 can operate under Level 5 autonomy (e.g., full driving automation), Level 4 autonomy (e.g., high driving automation), or Level 3 autonomy (e.g., conditional driving automation). As used herein the term “autonomous” includes both fully autonomous and semi-autonomous.

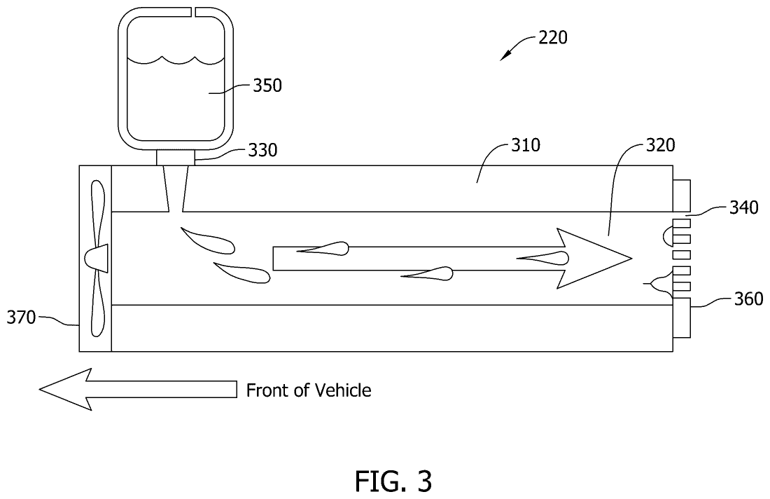

FIG. 3 is an illustration of one embodiment of the water temperature sensor 220. The water temperature sensor 220 includes a wind tunnel tube 310. The wind tunnel tube 310 forms a hollow path for airflow 320. The water temperature sensor 220 further includes a fluid valve 330 fluidly coupled to the wind tunnel tube 310, where a passage is defined through the fluid valve 330 and the wind tunnel tube 310. In various embodiments, the fluid valve 330 is coupled to the wind tunnel tube 310 by a mechanical attachment or a chemical bonding. In some embodiments, the fluid valve 330 is coupled to the wind tunnel tube 310 by being integrally formed with the wind tunnel tube 310. The fluid valve 330 is configured to dispense a fluid from a fluid reservoir 350 into the path for the airflow 320. In various embodiments, the fluid reservoir 350 is a windshield washer reservoir.

The water temperature sensor 220 further includes a wet-bulb temperature sensor 360 affixed to a vent 340. The vent 340 is located at the distal end of the wind tunnel tube 310. The vent 340 is coupled to the distal end of the wind tunnel tube 310. In various embodiments, the vent 340 is coupled to the wind tunnel tube 310 by a mechanical attachment or a chemical bonding. In some embodiments, the vent 340 is coupled with the wind tunnel tube 310 by being integrally formed with the wind tunnel tube 310 via a mechanism such as molding. The vent 340 and the wind tunnel tube 310 may be distinct pieces or formed as one single piece. The vent 340 facilitates contact between the fluid dispensed by the fluid valve 330 and the wet-blub temperature sensor 360. The wet-bulb temperature sensor 360 is affixed to the vent 340 to capture sensor data indicating a frost condition. In some embodiments, a fan 370 is affixed to the proximal end of the wind tunnel tube 310. The fan 370 is configured to control the airflow through the wind tunnel tube 310.

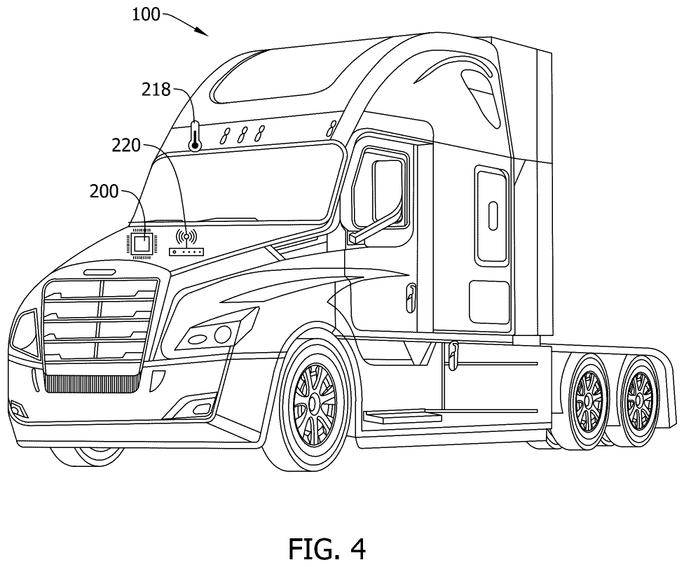

FIG. 4 is an illustration of the water temperature sensor 220 shown in FIG. 3 affixed on the autonomous vehicle 100 shown in FIGS. 1 and 2. water temperature sensor 220. Further, the water temperature sensor 220 is configured to communicatively couple to an autonomy computing system, such as autonomy computing system 200 shown in FIG. 2, of the autonomous vehicle 100. In various embodiments, the wind tunnel tube 310 extends to the exterior portion of the autonomous vehicle 100 so the path for the airflow 320 forms an airflow channel connecting the external environment of the autonomous vehicle 100 to the proximal end of the water temperature sensor 220.

In various embodiments, the autonomy computing system 200 is connected to the temperature sensor 218. The temperature sensor 218 includes, for example, a dry bulb temperature sensor on the autonomous vehicle. Autonomy computing system 200 is configured to receive sensor data from the dry-bulb temperature sensor. The sensor data from the dry-bulb temperature sensor includes temperature data corresponding to the ambient temperature of the environment surrounding the autonomous vehicle 100. The autonomy computing system 200 detects when the sensor data indicates a temperature below a temperature threshold. The temperature threshold includes a predetermined value corresponding to an ambient temperature where a frost condition begins to occur. Autonomy computing system 200 controls the fluid valve 330 to dispense a fluid into the airflow of the hollow path formed by the wind tunnel tube 310. For example, the autonomy computing system 200 controls the fluid valve 330 to dispense fluid at a rate to match an external humidity condition of the autonomous vehicle 100.

In various embodiments, the autonomy computing system 200 receives additional sensor data from the wet-bulb temperature sensor 360. The autonomy computing system processes the sensor data from the wet-bulb temperature sensor 360 to detect a frost condition from the additional sensor data. The frost condition corresponds to weather conditions that affect the performance of sensors 202 on the autonomous vehicle 100 resulting from frost forming on the sensors 202. In some embodiments, the autonomy computing system 200 computes a sensor degradation parameter for a sensor 202 on the autonomous vehicle 100 based on the frost condition. The degradation parameter corresponds to the reduced functionality of the sensor 202 resulting from the frost condition. In some embodiments, the autonomy computing system 200 is further configured to disengage the fluid valve 330 upon detection of the frost condition. The autonomy computing system 200 is further configured to control the fluid valve 330 to dispense fluid on a periodic time interval to verify the frost condition from further sensor data from the water temperature sensor 220.

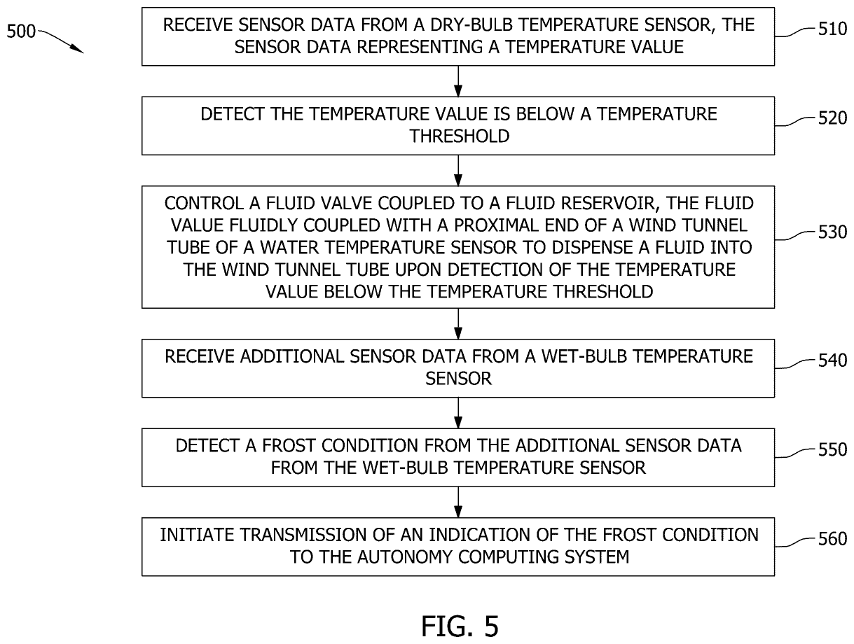

FIG. 5 is a flow diagram of an example method of detecting a frost condition with the water temperature sensor 220. The method 500 includes receiving 510 sensor data from a temperature sensor 218. The temperature sensor 218 includes, for example, a dry-bulb temperature sensor. In various embodiments, the sensor data from the dry-bulb temperature sensor represents a temperature value. Method 500 further includes detecting 520 that the temperature value is below a temperature threshold. The temperature threshold corresponds to a temperature with the possibility for a frost condition on a sensor 202 of the autonomous vehicle 100. Method 500 further includes controlling 530 a fluid valve coupled to a reservoir 350. The fluid valve 330 is coupled with a proximal end of the wind tunnel tube 310 of the water temperature sensor 220. Controlling 530 the fluid valve 330 includes dispensing a fluid into the wind tunnel tube 310 upon detection of the temperature value below the temperature threshold. Method 500 further includes receiving 540, additional sensor data from the wet-bulb temperature sensor 360 of the water temperature sensor 220. In various embodiments, method 500 further includes detecting 550 a frost condition from the additional sensor data from the wet-bulb temperature sensor 360. Method 500 further includes initiating 560 a transmission of an indication of the frost condition to the autonomy computing system 200. Method 500 may include additional, fewer, or alternative steps.

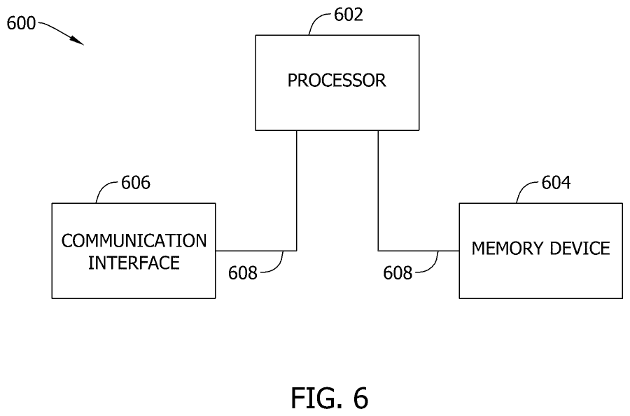

FIG. 6 is a block diagram of an example computing device 600. Computing device 600 includes a processor 602 and a memory device 604. The processor 602 is coupled to the memory device 604 via a system bus 608. The term “processor” refers generally to any programmable system including systems and microcontrollers, reduced instruction set computers (RISC), complex instruction set computers (CISC), application specific integrated circuits (ASIC), programmable logic circuits (PLC), and any other circuit or processor capable of executing the functions described herein. The above examples are example only, and thus are not intended to limit in any way the definition or meaning of the term “processor.”

In the example embodiment, the memory device 604 includes one or more devices that enable information, such as executable instructions or other data (e.g., sensor data), to be stored and retrieved. Moreover, the memory device 604 includes one or more computer readable media, such as, without limitation, dynamic random access memory (DRAM), static random access memory (SRAM), a solid state disk, or a hard disk. In the example embodiment, the memory device 604 stores, without limitation, application source code, application object code, configuration data, additional input events, application states, assertion statements, validation results, or any other type of data. The computing device 600, in the example embodiment, may also include a communication interface 606 that is coupled to the processor 602 via system bus 608. Moreover, the communication interface 606 is communicatively coupled to data acquisition devices.

In the example embodiment, processor 602 may be programmed by encoding an operation using one or more executable instructions and providing the executable instructions in the memory device 604. In the example embodiment, the processor 602 is programmed to select a plurality of measurements that are received from data acquisition devices.

In operation, a computer executes computer-executable instructions embodied in one or more computer-executable components stored on one or more computer-readable media to implement aspects of the disclosure described or illustrated herein. The order of execution or performance of the operations in embodiments of the disclosure illustrated and described herein is not essential, unless otherwise specified. That is, the operations may be performed in any order, unless otherwise specified, and embodiments of the disclosure may include additional or fewer operations than those disclosed herein. For example, it is contemplated that executing or performing a particular operation before, contemporaneously with, or after another operation is within the scope of aspects of the disclosure.

Some embodiments involve the use of one or more electronic processing or computing devices. As used herein, the terms “processor” and “computer” and related terms, e.g., “processing device,” and “computing device” are not limited to just those integrated circuits referred to in the art as a computer, but broadly refers to a processor, a processing device or system, a general purpose central processing unit (CPU), a graphics processing unit (GPU), a microcontroller, a microcomputer, a programmable logic controller (PLC), a reduced instruction set computer (RISC) processor, a field programmable gate array (FPGA), a digital signal processor (DSP), an application specific integrated circuit (ASIC), and other programmable circuits or processing devices capable of executing the functions described herein, and these terms are used interchangeably herein. These processing devices are generally “configured” to execute functions by programming or being programmed, or by the provisioning of instructions for execution. The above examples are not intended to limit in any way the definition or meaning of the terms processor, processing device, and related terms.

The various aspects illustrated by logical blocks, modules, circuits, processes, algorithms, and algorithm steps described above may be implemented as electronic hardware, software, or combinations of both. Certain disclosed components, blocks, modules, circuits, and steps are described in terms of their functionality, illustrating the interchangeability of their implementation in electronic hardware or software. The implementation of such functionality varies among different applications given varying system architectures and design constraints. Although such implementations may vary from application to application, they do not constitute a departure from the scope of this disclosure.

Aspects of embodiments implemented in software may be implemented in program code, application software, application programming interfaces (APIs), firmware, middleware, microcode, hardware description languages (HDLs), or any combination thereof. A code segment or machine-executable instruction may represent a procedure, a function, a subprogram, a routine, a subroutine, a module, a software package, a class, or any combination of instructions, data structures, or program statements. A code segment may be coupled to, or integrated with, another code segment or an electronic hardware by passing or receiving information, data, arguments, parameters, memory contents, or memory locations. Information, arguments, parameters, data, etc. may be passed, forwarded, or transmitted via any suitable means including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the disclosed functions may be embodied, or stored, as one or more instructions or code on or in memory. In the embodiments described herein, memory includes non-transitory computer-readable media, which may include, but is not limited to, media such as flash memory, a random access memory (RAM), read-only memory (ROM), erasable programmable read-only memory (EPROM), electrically erasable programmable read-only memory (EEPROM), and non-volatile RAM (NVRAM). As used herein, the term “non-transitory computer-readable media” is intended to be representative of any tangible, computer-readable media, including, without limitation, non-transitory computer storage devices, including, without limitation, volatile and non-volatile media, and removable and non-removable media such as a firmware, physical and virtual storage, CD-ROM, DVD, and any other digital source such as a network, a server, cloud system, or the Internet, as well as yet to be developed digital means, with the sole exception being a transitory propagating signal. The methods described herein may be embodied as executable instructions, e.g., “software” and “firmware,” in a non-transitory computer-readable medium. As used herein, the terms “software” and “firmware” are interchangeable and include any computer program stored in memory for execution by personal computers, workstations, clients, and servers. Such instructions, when executed by a processor, configure the processor to perform at least a portion of the disclosed methods.

As used herein, an element or step recited in the singular and proceeded with the word “a” or “an” should be understood as not excluding plural elements or steps unless such exclusion is explicitly recited. Furthermore, references to “one embodiment” of the disclosure or an “exemplary” or “example” embodiment are not intended to be interpreted as excluding the existence of additional embodiments that also incorporate the recited features. Likewise, limitations associated with “one embodiment” or “an embodiment” should not be interpreted as limiting to all embodiments unless explicitly recited.

Disjunctive language such as the phrase “at least one of X, Y, or Z,” unless specifically stated otherwise, is generally intended, within the context presented, to disclose that an item, term, etc. may be either X, Y, or Z, or any combination thereof (e.g., X, Y, and/or Z). Likewise, conjunctive language such as the phrase “at least one of X, Y, and Z,” unless specifically stated otherwise, is generally intended, within the context presented, to disclose at least one of X, at least one of Y, and at least one of Z.

The disclosed systems and methods are not limited to the specific embodiments described herein. Rather, components of the systems or steps of the methods may be utilized independently and separately from other described components or steps.

This written description uses examples to disclose various embodiments, which include the best mode, to enable any person skilled in the art to practice those embodiments, including making and using any devices or systems and performing any incorporated methods. The patentable scope is defined by the claims and may include other examples that occur to those skilled in the art. Such other examples are intended to be within the scope of the claims if they have structural elements that do not differ from the literal language of the claims, or if they include equivalent structural elements with insubstantial differences form the literal language of the claims.

What is claimed is:

1. A water temperature sensor for an autonomous vehicle, the water temperature sensor comprising:
a wind tunnel tube forming a hollow path for an airflow;
a fluid valve coupled to a fluid reservoir, the fluid valve fluidly coupled with a proximal end of the wind tunnel tube to dispense a fluid into the airflow of the hollow path;
a vent coupled to a distal end of the wind tunnel tube; and
a wet-bulb temperature sensor affixed to the vent.

2. The water temperature sensor of claim 1, wherein the fluid has a freezing point less than 32° Fahrenheit.

3. The water temperature sensor of claim 1, wherein the fluid reservoir is a windshield washer reservoir.

4. The water temperature sensor of claim 1 further comprising a fan coupled to the wind tunnel tube, wherein the fan is disposed proximal to the fluid valve on the hollow path for the airflow.

5. The water temperature sensor of claim 4, wherein the fan is configured to modify the airflow in response to an external weather condition observed by the autonomous vehicle.

6. The water temperature sensor of claim 1, wherein the fluid valve is configured to dispense the fluid based on a humidity level of an external environment in which the autonomous vehicle is operating.

7. The water temperature sensor of claim 1 further comprising a processor connected to a memory storing computer executable instructions, the processor, upon executing the computer executable instructions, configured to:
receive sensor data from the wet-bulb temperature sensor;
identify a frost condition from the sensor data; and
transmit an indication of the frost condition to the autonomous vehicle.

8. An autonomous vehicle comprising:
a water temperature sensor, the water temperature sensor comprising:
a wind tunnel tube forming a hollow path for an airflow,
a fluid valve coupled to a fluid reservoir, the fluid valve fluidly coupled with a proximal end of the wind tunnel tube to dispense a fluid into the airflow of the hollow path,
a vent coupled to a distal end of the wind tunnel tube, and
a wet-bulb temperature sensor affixed to the vent;
a dry-bulb temperature sensor; and
an autonomy computing system comprising a processor connected to a memory storing computer executable instructions, the processor, upon executing the computer executable instructions, configured to:
receive sensor data from the dry-bulb temperature sensor on the autonomous vehicle,
detect the sensor data is below a temperature threshold,
control the fluid valve on the water temperature sensor to dispense a fluid into the airflow of the hollow path,
receive additional sensor data from the water temperature sensor,
detect a frost condition from the additional sensor data, and
initiate transmission of an indication of the frost condition to the autonomy computing system.

9. The autonomous vehicle of claim 8, wherein the fluid has a freezing point less than 32°Fahrenheit.

10. The autonomous vehicle of claim 8, wherein the fluid valve is configured to dispense the fluid at a rate to match an external humidity condition.

11. The autonomous vehicle of claim 8 further comprising an airflow channel connecting an external environment to the proximal end of the water temperature sensor.

12. The autonomous vehicle of claim 8, wherein the autonomy computing system is further configured to control the fluid valve to dispense the fluid on a periodic time interval to verify the frost condition from further sensor data from the water temperature sensor.

13. The autonomous vehicle of claim 8, wherein the processor is further configured to compute a sensor degradation parameter for a sensor on the autonomous vehicle based on the frost condition.

14. The autonomous vehicle of claim 8, wherein the processor is further configured to disengage the fluid valve upon the detection of the frost condition.

15. A computer-implemented method for detecting a frost condition on an autonomous vehicle, the method implemented by an autonomy computing system of the autonomous vehicle, the autonomy computing system including a processor and a memory storing executable instructions, the computer-implemented method comprising:
receiving sensor data from a dry-bulb temperature sensor, the sensor data representing a temperature value;
detecting the temperature value is below a temperature threshold;
controlling a fluid valve coupled to a fluid reservoir, the fluid valve fluidly coupled with a proximal end of a wind tunnel tube of a water temperature sensor to dispense a fluid into the wind tunnel tube upon detection of the temperature value below the temperature threshold;
receiving additional sensor data from a wet-blub temperature sensor;
detecting a frost condition from the additional sensor data from the wet-bulb temperature sensor; and
initiating transmission of an indication of the frost condition to the autonomy computing system.

16. The computer-implemented method of claim 15, wherein controlling a fluid valve further comprises dispensing a fluid with a freezing point less than 32° Fahrenheit.

17. The computer-implemented method of claim 15, wherein controlling the fluid valve further comprises dispensing the fluid from a windshield washer reservoir.

18. The computer-implemented method of claim 15, wherein controlling the fluid valve further comprises dispensing the fluid based on an external humidity condition.

19. The computer-implemented method of claim 15, further comprising controlling a fan on the water temperature sensor to adjust an airflow in the wind tunnel tube.

20. The computer-implemented method of claim 15, further comprising terminating the dispensation of the fluid from the fluid valve upon detection of the frost condition.
